(19) United States
(12) Patent Application Publication
(10) Pub. No.: US 2026/0157975 A1
(43) Pub. Date: Jun. 11, 2026

(54) SILOXANE NANOCOMPOSITE MATERIAL AND ITS PREPARATION METHOD AND APPLICATION

(71) Applicants: Kai Yao, Shenzhen (CN); Haopeng Zhang, Shenzhen (CN)

(72) Inventors: Haopeng Zhang, Shenzhen (CN); Kai Yao, Shenzhen (CN); Guangtao Huang, Shenzhen (CN); Binbin Gui, Shenzhen (CN); Huiqiao Han, Shenzhen (CN); Bin Kong, Shenzhen (CN); Dahui Xue, Shenzhen (CN); Xinyu Gao, Shenzhen (CN); Yijia Sun, Shenzhen (CN); Yi Lin, Shenzhen (CN); Tao Jiang, Shenzhen (CN); Yiwei Zhang, Shenzhen (CN); Chunyang Liu, Shenzhen (CN)

(21) Appl. No.: 19/411,382

(22) Filed: Dec. 8, 2025

(30) Foreign Application Priority Data
Dec. 10, 2024 (CN) 202411803692.3

(51) Int. Cl. A61K 9/70; A61K 9/00; A61K 31/352; A61K 33/38; B82Y 5/00

(52) U.S. Cl. CPC A61K 9/7007; A61K 9/0085; A61K 31/352; A61K 33/38; B82Y 5/00

(57) ABSTRACT

A siloxane nanocomposite material, and its preparation method and application are provided, and relate to the field of biochemistry technology. The preparation method includes: mixing nano-silver, quercetin and a solvent uniformly to obtain a suspension; and adding a polysiloxane film to the suspension and stirring for reaction to obtain the siloxane nanocomposite material. A weight ratio of the nano-silver to the polysiloxane film is 1:10-20, a weight-volume ratio of the polysiloxane film to the solvent is 0.2-0.25 g:30 mL, and an added amount of the quercetin is 3 mg per milliliter of the solvent. The preparation method is simple; the key raw materials including siloxane, nano-silver, and quercetin, are easily available and inexpensive, resulting in low production costs. The siloxane nanocomposite material exhibits strong anti-adhesion, broad-spectrum antibacterial, and anti-inflammatory properties, and effectively promotes temporal-muscle repair after decompressive craniectomy, thereby improving patient prognosis quality.

### CROSS-REFERENCE TO RELATED APPLICATION

This application claims priority to Chinese Patent Application No. 202411803692.3, filed Dec. 10, 2024, which is herein incorporated by reference in its entirety.

### TECHNICAL FIELD

The disclosure relates to the field of biochemistry technology, and more particularly to a siloxane nanocomposite material, and its preparation method and application.

### BACKGROUND

In the field of neurosurgery, when acute central nervous system diseases such as stroke and traumatic brain injury occur, they are usually accompanied by a rapid increase in intracranial pressure. High intracranial pressure can lead to cerebral ischemia and necrosis, and even brain herniation, which endangers the life of patient. Decompressive craniectomy, a neurosurgical procedure involving the removal of part of the skull to accommodate brain swelling, is often followed by temporal muscle damage and adhesion formation between the temporal muscle, the bone flap, and the dura mater. This is a common but very difficult clinical problem, which not only significantly increases the cost, difficulty, and postoperative complications of subsequent cranioplasty, such as causing inflammatory reactions, epilepsy, and infections induced by cerebrospinal fluid leakage that endanger life, but also severely changes the facial appearance of patient and affects the normal functions of the temporal muscle, such as chewing, and even cause severe pain. All these greatly reduce the prognosis quality of patient and bring a heavy mental and economic burden to patients and their families.

At the same time when this clinical phenomenon urgently needs to be solved, there are almost no materials on the market that can directly prevent or solve the above problems. Common artificial dura mater materials used in clinical practice, including polytetrafluoroethylene (PTFE) and acellular dermal matrix (ADM), are very expensive, making patients hesitate, and may provide some characteristics that are not needed for this application, such as affecting the sealing ability around the suture and the tissue growth into the surface. On the other hand, these materials are also prone to cause infections and inflammatory reactions when placed in the skull, and most importantly, these commercially available biological dura mater materials cannot effectively prevent infections, adhesion formation, and promote temporal muscle repair.

### SUMMARY

In view of the foregoing, the disclosure aims to provide a siloxane nanocomposite material and its preparation method and application.

In order to achieve the above purpose, the disclosure provides the following technical solutions.

A first technical solution of the disclosure is a preparation method of a siloxane nanocomposite material, including: mixing nano-silver, quercetin and a solvent uniformly to obtain a suspension; and adding a polysiloxane film to the suspension and stirring for reaction to obtain the siloxane nanocomposite material.

A weight ratio of the nano-silver to the polysiloxane film is 1:10-20, a weight-volume ratio of the polysiloxane film to the solvent is 0.2-0.25 grams (g):30 milliliters (mL), and an added amount of the quercetin is 3 milligrams (mg) per mL of the solvent.

A second technical solution of the disclosure is the siloxane nanocomposite material prepared by the preparation method.

A third technical solution of the disclosure is an application of the siloxane nanocomposite material, including: applying the siloxane nanocomposite material in the repair of the temporal muscle after decompressive craniectomy.

The disclosure further provides an application of the siloxane nanocomposite material, including: applying the siloxane nanocomposite material in preparing a drug for temporal muscle repair after decompressive craniectomy.

The disclosure has the following technical effects.

The preparation method of the siloxane nanocomposite material provided by the disclosure is simple. The core components of the raw materials, such as the siloxane, the nano-silver, and the quercetin, are easily available and inexpensive, resulting in low production costs.

The siloxane nanocomposite material provided by the disclosure has advantages of good anti-adhesion, broad-spectrum antibacterial properties, anti-inflammatory effects, and promoting the repair of the temporal muscle after decompressive craniectomy, and improve the prognosis of patients and reduce the burden on families and society.

The siloxane nanocomposite material provided by the disclosure has good slow-release properties, which can further enhance the repair effect of the temporal muscle after decompressive craniectomy.

### BRIEF DESCRIPTION OF DRAWINGS

In order to more clearly illustrate embodiments of the disclosure or the technical solutions in the related art, a brief introduction will be given to the drawings required for the embodiments. It is apparent that the drawings described below are only some embodiments of the disclosure. For those skilled in the art, other drawings can be obtained based on these drawings without creative labor.

FIG. 1 illustrates a chemical structure of polysiloxane of the disclosure.

FIG. 2 illustrates a schematic diagram of components of a siloxane nanocomposite material and an application of the siloxane nanocomposite material in an animal decompressive craniectomy model of the disclosure, where AQS represents the siloxane nanocomposite material.

FIG. 3 illustrates a scanning electron microscope image of the siloxane nanocomposite material according to an effect verification example of the disclosure.

FIGS. 4A-4D illustrate two-dimensional (2D) and three-dimensional (3D) structures of the polysiloxane and the siloxane nanocomposite material under an atomic force microscope according to the effect verification example, where FIGS. 4A and 4B show 2D and 3D morphologies of the polysiloxane, and FIGS. 4C and 4D show 2D and 3D morphologies of the siloxane nanocomposite material.

FIGS. 5A-5C illustrate hydrophobic angles of a polytetrafluoroethylene material, the siloxane nanocomposite material, and the original polysiloxane film according to the effect verification example, where FIG. 5A represents the polytetrafluoroethylene material, FIG. 5B represents the original polysiloxane film, and FIG. 5C represents the siloxane nanocomposite material.

FIG. 6 illustrates antibacterial ability of different materials against Escherichia coli and Staphylococcus aureus according to the effect verification example.

FIG. 7A illustrates a slow-release ability detection result of silver in the siloxane nanocomposite material according to the effect verification example, where Ag represents silver, and FIG. 7B illustrates a slow-release ability detection result of quercetin in the siloxane nanocomposite material according to the effect verification example.

FIGS. 8A-8F illustrate immunofluorescence images of expression levels of pro-inflammatory factor interleukin-6 (IL-6), anti-inflammatory factor interleukin-10 (IL-10), and proliferating cell nuclear antigen (PCNA) in the damaged temporal muscle in the animal model according to the effect verification example, where FIGS. 8A and 8B respectively represent the levels of the IL-6 on days 7 and 14 after rat modeling, FIGS. 8C and 8D respectively represent the levels of the IL-10 on days 7 and 14 after rat modeling, and FIGS. 8E and 8F respectively represent the levels of the PCNA on days 7 and 14 after rat modeling.

FIG. 9 illustrates effects of the siloxane nanocomposite material on the temporal muscle on days 7 and 14 postoperative in a rat decompressive craniectomy model according to the effect verification example.

### DETAILED DESCRIPTION OF EMBODIMENTS

Various exemplary embodiments of the disclosure are now described in detail, which should not be construed as limiting the disclosure, but rather as a more detailed description of certain aspects, features, and embodiments of the disclosure.

It should be understood that terms described in the disclosure are only for describing specific embodiments and are not intended to limit the disclosure. In addition, for the numerical range in the disclosure, it should be understood that each intermediate value between the upper and lower limits of the range is also specifically disclosed. Any intermediate value within any stated value or range, as well as any smaller range between any other stated value or intermediate value within the range, are also included in the disclosure. These smaller upper and lower limits can be independently included or excluded within the range.

Unless otherwise specified, all technical and scientific terms used herein have the same meanings as those commonly understood by those skilled in the art of the disclosure. Although the disclosure only describes exemplary methods and materials, any methods and materials similar or equivalent to those described herein may also be used in the implementation or testing of the disclosure. All references mentioned herein are incorporated by reference to disclose and describe methods and/or materials related to the mentioned references. In case of conflict with any incorporated literature, the content of this specification shall prevail.

Various improvements and variations can be made to the embodiments of the specification of the disclosure without departing from the scope or spirit of the disclosure, which will be apparent to those skilled in the art. Other embodiments obtained from the specification of the disclosure will be apparent to those skilled in the art. The specification and embodiments of the disclosure are only exemplary.

The terms “including”, “possessing”, “containing”, etc. used herein are all open-ended terms, meaning they include but are not limited to.

Room temperature in the disclosure is in a range of 15-35 degrees Celsius (° C.).

A first technical solution of the disclosure is a preparation method of a siloxane nanocomposite material, including: mixing nano-silver, quercetin and a solvent uniformly to obtain a suspension; and adding a polysiloxane film to the suspension and stirring for reaction to obtain the siloxane nanocomposite material.

A weight ratio of the nano-silver to the polysiloxane film is 1:10-20, a weight-volume ratio of the polysiloxane film to the solvent is 0.2-0.25 g:30 mL, an added amount of the quercetin is 3 mg per mL of the solvent.

In the disclosure, a preparation method of the polysiloxane film is derived from “Preparation of Robust, Room-Temperature Self-Healable and Recyclable Polysiloxanes Based on Hierarchical Hard Domains,” by Pengyuan Lai et al., published in Advanced Engineering Materials. The polysiloxane material actually used was gifted by the authors of the article.

In some embodiments, a particle size of the nano-silver is 100 nanometers (nm).

In some embodiments, the solvent is deionized water, and the stirring for reaction includes: stirring in the dark at normal temperature and pressure for 12-24 hours with a stirring speed of 400-500 revolutions per minute (rpm).

In some embodiments, the adding a polysiloxane film to the suspension and stirring for reaction to obtain the siloxane nanocomposite material includes: adding the polysiloxane film to the suspension and stirring for reaction to obtain a reaction product, and drying the reaction product to obtain the siloxane nanocomposite material, where the drying the reaction product includes: air-drying the reaction product in the dark at room temperature for 0.5-1 hour.

In the disclosure, the nano-silver and the quercetin are loaded onto the polysiloxane material through the stirring and reaction, endowing the siloxane nanocomposite material with slow-release properties. The loaded nano-silver and quercetin provide the siloxane nanocomposite material with antibacterial and anti-inflammatory effects, and significantly enhance the hydrophobicity of the polysiloxane material. The hydrophobicity is more conducive to preventing adhesions between the temporal muscle, the bone flap, and the dura mater, thereby promoting the healing of the temporal muscle and reducing the occurrence of complications.

A second technical solution of the disclosure is the siloxane nanocomposite material obtained by the above preparation method.

A third technical solution of the disclosure is an application of the siloxane nanocomposite material, including: applying the siloxane nanocomposite material in the repair of the temporal muscle after decompressive craniectomy.

In order to better understand the disclosure, the content of the disclosure will be further explained in conjunction with the embodiments below, but the content of the disclosure is not limited to the embodiments below.

Unless otherwise specified, the raw materials and reagents used in the embodiments can be obtained through commercial channels.

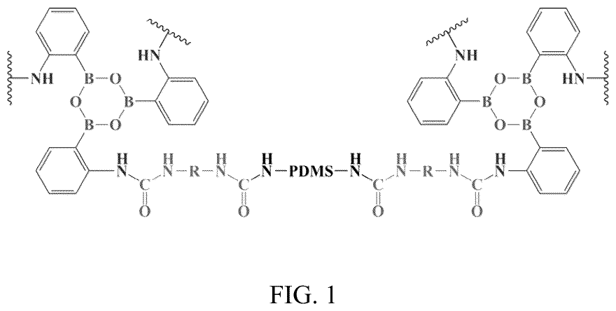

In the embodiments, a preparation method of the polysiloxane film is derived from “Preparation of Robust, Room-Temperature Self-Healable and Recyclable Polysiloxanes Based on Hierarchical Hard Domains,” by Pengyuan Lai et al., published in Advanced Engineering Materials. The polysiloxane material actually used was gifted by the authors of the article. Specifically, the preparation method of the polysiloxane includes the following steps. Bis(3-aminopropyl) terminated polydimethylsiloxane (APDMS, 3 g, 10 millimoles) is dissolved with 10 mL of dry tetrahydrofuran in a 50 mL round-bottom flask to obtain an APDMS solution. 4,4′-methylene diphenyl diisocyanate (MDI) and isophorone diisocyanate (IPDI) are dissolved in tetrahydrofuran at a stoichiometric ratio of the APDMS, the MDI, the IPDI, and the 2-aminophenylboronic acid being 1:0.6:1.4:2 to obtain a mixed solution, and the mixed solution is added dropwise to the APDMS solution, followed by stirring for 5 hours under nitrogen and ice bath conditions to undergo a reaction to obtain an APDMS-NCO solution, where NCO represents an isocyanate group. The APDMS-NCO solution is stored for later use. 2-aminophenylboronic acid (0.274 g, 20 millimoles) is dissolved in 10 mL of tetrahydrofuran and then stirred for 5 minutes to obtain a 2-aminophenylboronic acid solution, a small amount of insoluble substances are removed from the 2-aminophenylboronic acid solution through a microporous filter membrane to obtain a filtered solution, and the filtered solution is added to the APDMS-NCO solution followed by stirring for 5 hours to obtain an APDMS-MDI-IPDI-B solution, where B represents the 2-aminophenylboronic acid. Reduced-pressure distillation is performed on the APDMS-MDI-IPDI-B solution to remove excess tetrahydrofuran, and then the APDMS-MDI-IPDI-B solution is poured into a Teflon mold (i.e., a PTFE mold), followed by drying continuously at room temperature for 24 hours to obtain an APDMS-MDI-IPDI-B film. The APDMS-MDI-IPDI-B film is dried at room temperature in a vacuum oven for 12 hours to obtain the polysiloxane film. The structure of the polysiloxane film is shown in FIG. 1.

### Embodiment 1

The siloxane nanocomposite material is prepared through the following steps.

0.2 g of the polysiloxane film is weighed using a precision balance with a sensitivity of no less than milligram level for later use. Then, nano-silver powder with a particle size of approximately 100 nm (a weight ratio of the nano-silver to the polysiloxane is 1:10) and quercetin (an added amount of the quercetin is 3 mg per mL of deionized water) are added to 30 mL of the deionized water followed by treating with an ultrasonic machine for 45 minutes to evenly disperse the nano-silver powder and the quercetin in the deionized water to obtain a suspension. Subsequently, the polysiloxane film is added to the suspension, and then a sterilized clean magnetic stirrer bar is added to the suspension, followed by stirring in the dark under normal temperature and pressure (ambient temperature and pressure) for 24 hours using a magnetic stirrer at a speed of 400 rpm to obtain a mixed film as a reaction product. Finally, the mixed film is taken out and air-dried in the dark at room temperature in a mold for 1 hour to obtain the siloxane nanocomposite material.

### Effect Verification Example

The distribution of the nano-silver on the siloxane nanocomposite material is shown in FIG. 3, the scanning electron microscope result shows that the distribution of the nano-silver on the siloxane nanocomposite material is uniform across the surface without aggregation, which is beneficial for the subsequent antibacterial properties of the siloxane nanocomposite material. The nano-silver particles on the siloxane nanocomposite material are spherical, with an average diameter of approximately 100 nm as measured.

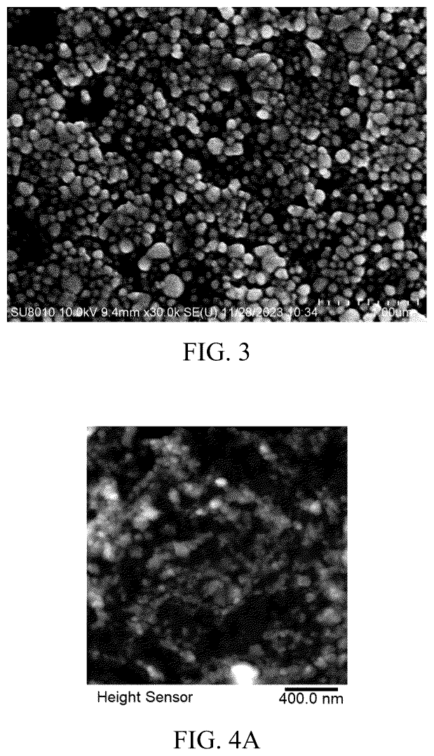

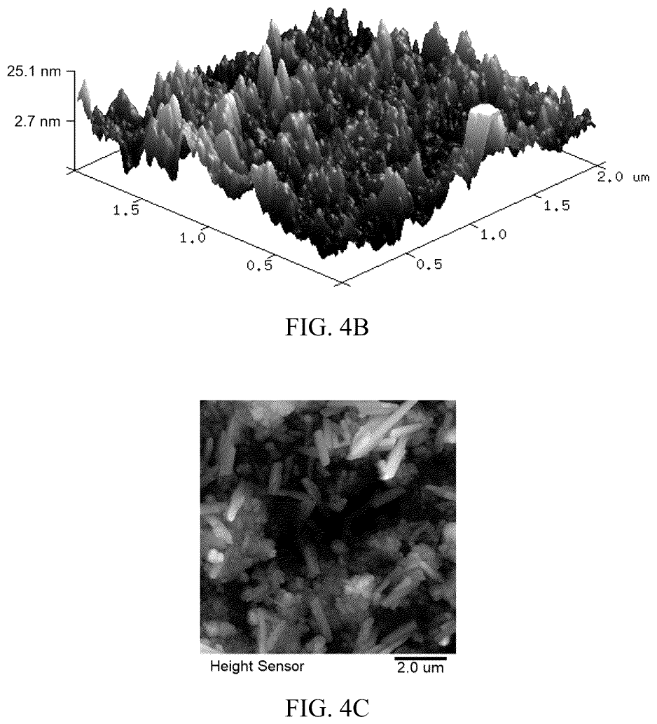

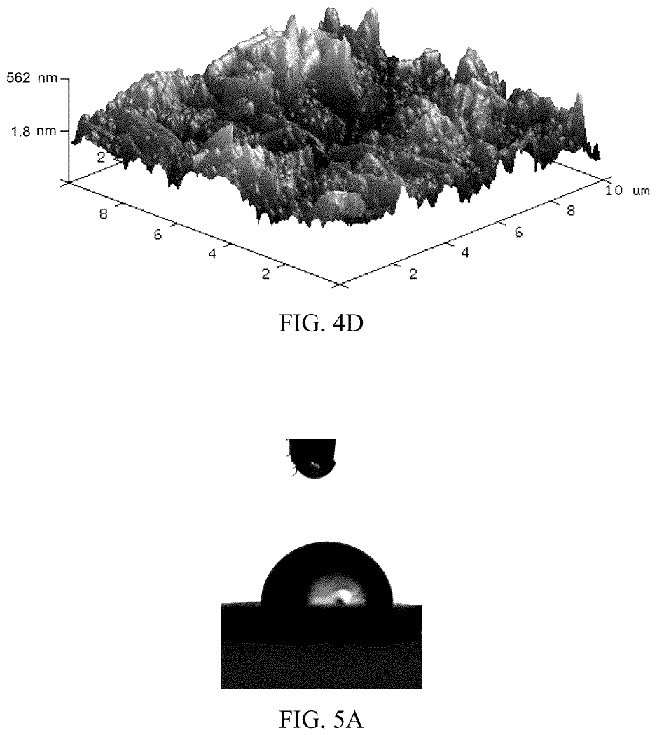

The structure of the siloxane nanocomposite material is shown in FIGS. 4A-4D. FIGS. 4A and 4B show the 2D and 3D morphologies of the polysiloxane material observed under the atomic force microscopy, respectively. FIGS. 4C and 4D respectively show 2D and 3D morphologies of the siloxane nanocomposite material. The quercetin and the nano-silver are uniformly distributed on the siloxane nanocomposite material, consistent with the scanning electron microscopy results. In FIG. 4C, the spherical objects are silver nanoparticles, and the rod-like objects are the quercetin. Compared with the original polysiloxane, the atomic force microscopy images indicate that the nano-silver and the quercetin have been successfully loaded onto the siloxane nanocomposite material.

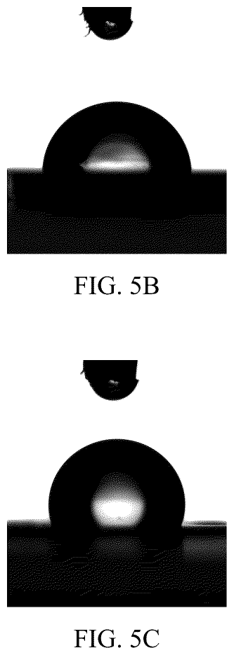

The anti-adhesion property of the siloxane nanocomposite material is mainly due to its excellent hydrophobicity. After decompressive craniectomy, the siloxane nanocomposite material is placed between the dura mater and the damaged temporal muscle to form an isolation barrier. The good hydrophobicity helps prevent adhesions between the temporal muscle, the bone flap, and the dura mater, thereby promoting the healing of the temporal muscle and reducing the occurrence of complications. FIG. 5C shows that the detection result of the hydrophobic angle of the siloxane nanocomposite material is about 108.88°, which indicates that the hydrophobic degree of the siloxane nanocomposite material is better than the polytetrafluoroethylene material (89.41°, FIG. 5A) and the original polysiloxane material (96.02°, FIG. 5B). The acellular matrix material is hydrophilic and does not possess hydrophobicity, making it incomparable to the siloxane nanocomposite material.

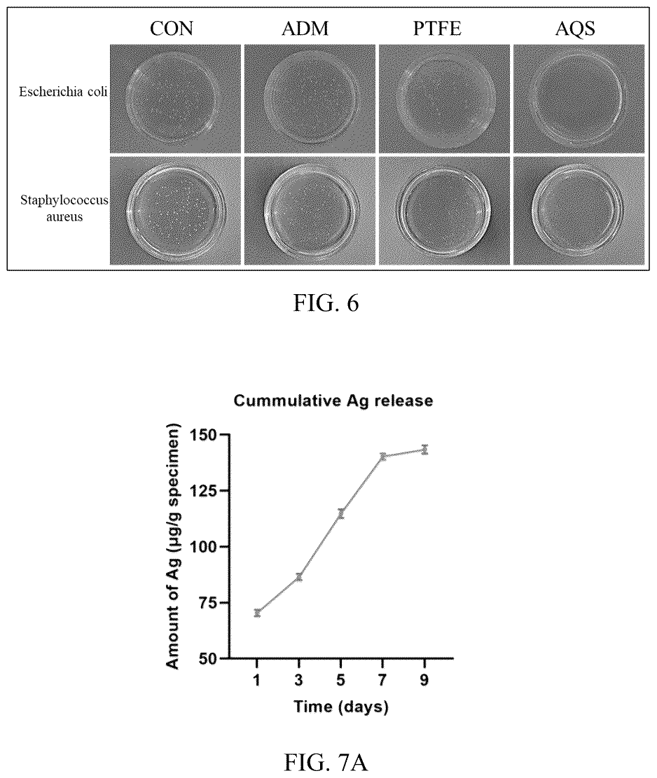

The broad-spectrum antibacterial properties of the siloxane nanocomposite material are highly beneficial for inhibiting bacterial growth in wounds and improving wound healing efficiency. This is mainly because the nano-silver interferes with bacterial reproduction and disrupts bacterial structures to kill bacteria, thereby providing a more suitable microenvironment for the repair of the temporal muscle. To evaluate its antibacterial capacity, two standard drug-resistant strains (Escherichia coli and Staphylococcus aureus), which are the most common in surgical infections, are selected. A control group (CON) without any material added is used for comparison. Different materials are co-cultured with the Escherichia coli or the Staphylococcus aureus under the same bacterial dilution conditions. The growth of bacterial colonies on the plates for each group is shown in FIG. 6 (where CON represents the control group; ADM represents acellular dermal matrix; PTFE represents polytetrafluoroethylene; and AQS represents the siloxane nanocomposite material). The siloxane nanocomposite material exhibits very strong antibacterial activity. After quantifying the number of bacterial colonies, it is found that the siloxane nanocomposite material has a bacterial killing rate of over 99% for the Escherichia coli and over 90% for the Staphylococcus aureus. Meanwhile, the commonly used PTFE and ADM on the market do not possess the broad-spectrum antibacterial properties of the siloxane nanocomposite material.

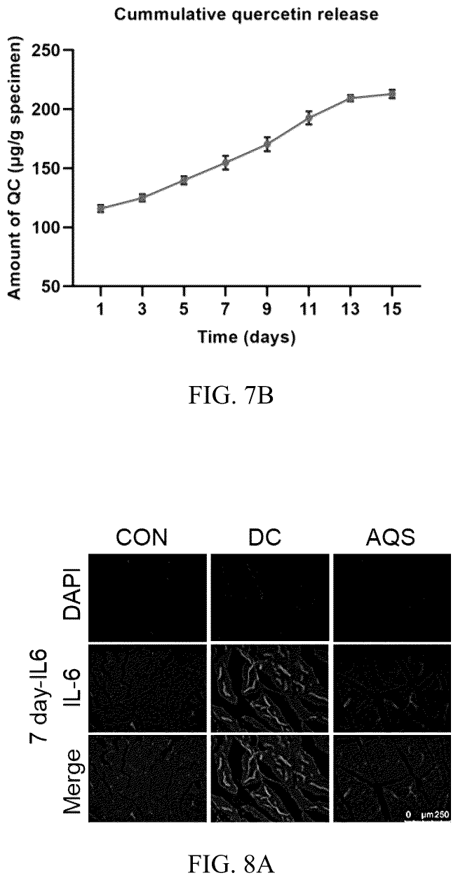

The siloxane nanocomposite material has the ability to slowly release the nano-silver and drugs, which is of great significance for sustained antibacterial and anti-inflammatory effects and for promoting temporal muscle repair over a period of time after decompressive craniectomy. To study the release of the silver and the quercetin, the siloxane nanocomposite material is immersed in phosphate-buffered saline (PBS, pH=7.4) at 37° C. The supernatant is collected every other day and replaced with an equal amount of fresh PBS. The collected supernatant is measured for absorbance using a microplate reader, and the concentration of the quercetin at different time points is calculated based on the absorbance. Meanwhile, the supernatant is mixed with an equal volume of aqua regia and dissolved to obtain a mixture. The mixture is then diluted to a fixed volume with distilled water and analyzed using inductively coupled plasma optical emission spectrometry (ICP-OES) to determine the concentration of the silver. The release result of the silver is shown in FIG. 7A, where the release of the silver from the siloxane nanocomposite material increases sharply from day 1 to day 7, then slows down significantly from day 7, and reaches saturation on day 9. The release result of the quercetin is shown in FIG. 7B, where the release of the quercetin increases gradually from day 1 to day 13. The release rate slows down from day 13 and reaches saturation on day 15. The above results indicate that the siloxane nanocomposite material, loaded with the silver and the quercetin, has good slow-release properties, which can enhance the actual efficacy of antibacterial and anti-inflammatory effects, thereby further promoting the repair of the temporal muscle after decompressive craniectomy.

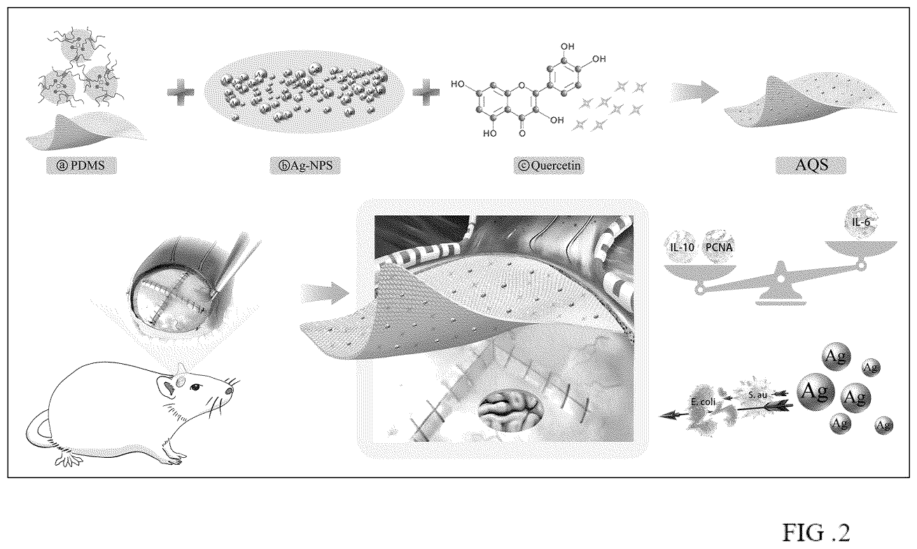

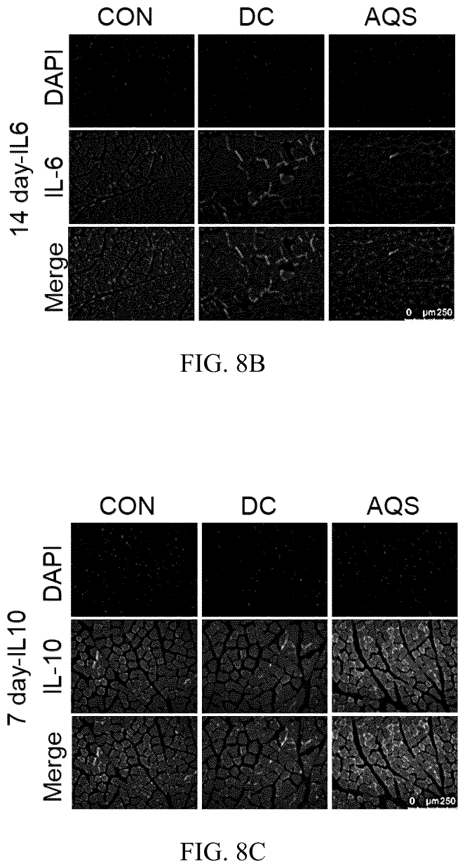

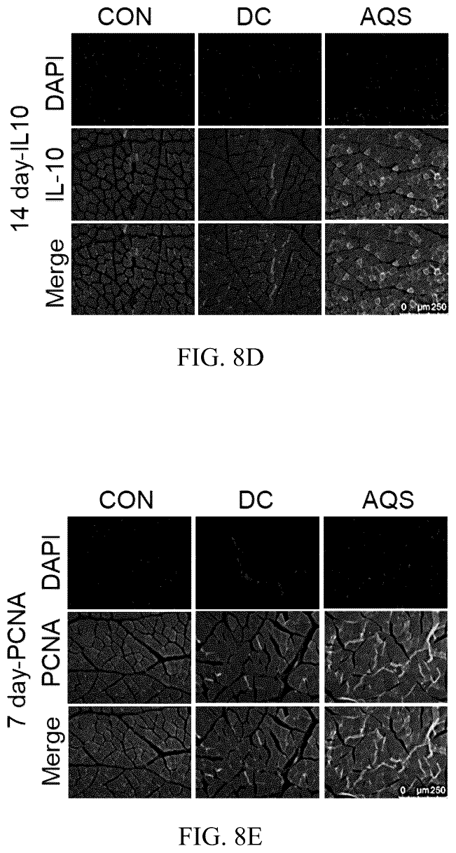

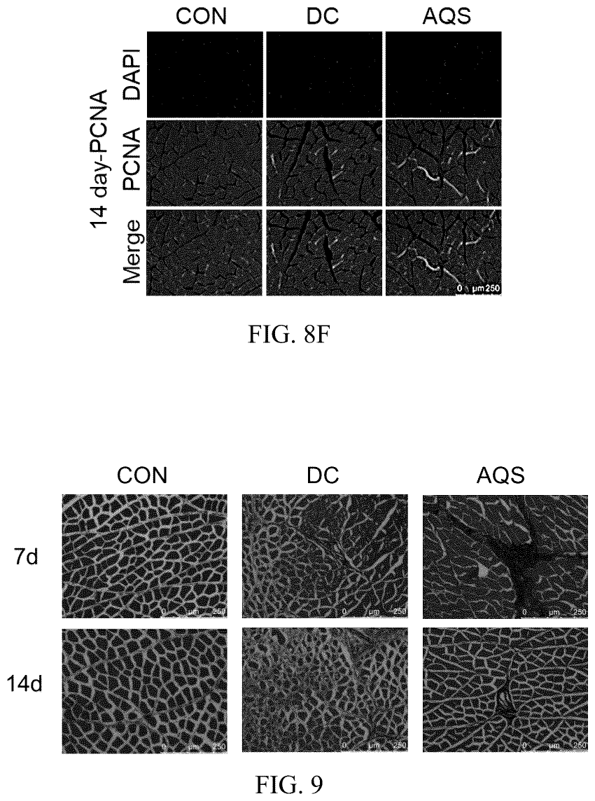

The anti-inflammatory and pro-healing properties of the siloxane nanocomposite material are primarily achieved by the quercetin, which increases the expression of anti-inflammatory factor IL-10 and pro-healing factor PCNA, and decreases the expression of pro-inflammatory factor IL-6. To verify the actual performance of the siloxane nanocomposite material, the siloxane nanocomposite material is applied to a rat decompressive craniectomy model, with the relevant ethics approved by the Ethics Committee of Harbin Medical University. The experiment was conducted as follows: First, the appropriate cranial bone of the rat is removed and the temporal muscle is separated. To align with clinical practice, an appropriate incision is made in the exposed dura mater, and then the dura mater is then sutured. Subsequently, the siloxane nanocomposite material is placed between the dura mater, and the temporal muscle and the bone edge (as shown in FIG. 2). The separated temporal muscle is then repositioned and the skin is sutured. On postoperative days 7 and 14, the rats are euthanized, and the temporal muscle at the modeling site is sectioned and examined for the expression levels of IL-6, IL-10, and PCNA using immunofluorescence (CON: normal control; DC: decompressive craniectomy modeling only; AQS: decompressive craniectomy modeling+the siloxane nanocomposite material). FIGS. 8A and 8B show the levels of IL-6 on days 7 and 14 after rat modeling, FIGS. 8C and 8D show the levels of IL-10 on days 7 and 14 after rat modeling, and FIGS. 8E and 8F show the levels of PCNA on days 7 and 14 after rat modeling. These results indicate that the siloxane nanocomposite material regulates the inflammatory response by downregulating the IL-6 level and upregulating the IL-10 level, and improves the expression of the pro-healing factor PCNA to create a more favorable environment for temporal muscle repair, avoiding excessive inflammatory reactions that could hinder the healing process. The above properties are not possessed by the commonly used clinical materials (PTFE, ADM).

The ultimate therapeutic effect of the siloxane nanocomposite material on the temporal muscle in the rat decompressive craniectomy model is shown in FIG. 9. Temporal-muscle tissue sections from the modeled rats are subjected to Masson's trichrome staining to observe collagen-rich areas on postoperative days 7 and 14 in the different experimental groups (collagen is stained blue, muscle fibers are stained red; CON: normal control; DC: decompressive craniectomy modeling only; AQS: decompressive craniectomy+the siloxane nanocomposite material). The results confirm that AQS exerts multiple beneficial actions: anti-adhesion, antibacterial, anti-inflammatory, and pro-healing. On postoperative day 7, the DC group exhibits an increased collagen-matrix area compared with the CON group, but the effect in the DC group is markedly inferior to the effect in the AQS group. On postoperative day 14, the collagen-rich region in the DC group is still expanding, whereas the AQS group shows almost complete muscle reconstitution with a histological morphology close to that of the CON group, demonstrating that the siloxane nanocomposite material significantly accelerates temporal-muscle remodeling and healing after decompressive craniectomy.

In summary, when the siloxane nanocomposite material is used during decompressive craniectomy, the siloxane nanocomposite material creates an optimal healing microenvironment for the injured temporal muscle by preventing adhesions, exerting broad-spectrum antibacterial action, suppressing inflammation, and promoting healing performance, thereby robustly promoting temporal-muscle repair after surgery. While improving patient prognosis and reducing the burden on patients, families, and society, the siloxane nanocomposite material also significantly lowers healthcare costs, making it well suited for widespread clinical adoption and large-scale application.

The above is only the exemplary embodiment of the disclosure. It should be pointed out that for those skilled in the art, several improvements and embellishments can be made without departing from the principles of the disclosure, and these improvements and embellishments should also be considered as the scope of protection of the disclosure.

What is claimed is:

1. An application of a siloxane nanocomposite material, comprising:
applying the siloxane nanocomposite material in preparing a drug for temporal muscle repair after decompressive craniectomy,
wherein the siloxane nanocomposite material is prepared through the following steps:
mixing nano-silver, quercetin and a solvent uniformly to obtain a suspension; and
adding a polysiloxane film to the suspension and stirring for reaction to obtain the siloxane nanocomposite material;
wherein a weight ratio of the nano-silver to the polysiloxane film is 1:10, a weight-volume ratio of the polysiloxane film to the solvent is 0.2 grams (g):30 milliliters (mL), an added amount of the quercetin is 3 milligrams (mg) per mL of the solvent, and the solvent is deionized water; and
wherein the polysiloxane film is prepared through the following steps:
dissolving 10 millimoles (mmol) of bis(3-aminopropyl) terminated polydimethylsiloxane (APDMS) in 10 mL of dry tetrahydrofuran to obtain an APDMS solution;
dissolving 4,4′-methylene diphenyl diisocyanate (MDI) and isophorone diisocyanate (IPDI) in tetrahydrofuran to obtain a mixed solution, and adding the mixed solution dropwise to the APDMS solution, followed by stirring for 5 hours under nitrogen and ice bath conditions to obtain an APDMS-NCO solution;
dissolving 20 mmol of 2-aminophenylboronic acid in 10 mL of tetrahydrofuran and stirring for 5 minutes to obtain a 2-aminophenylboronic acid solution, removing insoluble substances from the 2-aminophenylboronic acid solution through a microporous filter membrane to obtain a filtered solution, and adding the filtered solution to the APDMS-NCO solution followed by stirring for 5 hours to obtain an APDMS-MDI-IPDI-B solution; and
performing reduced-pressure distillation on the APDMS-MDI-IPDI-B solution to remove excess tetrahydrofuran, and then pouring the APDMS-MDI-IPDI-B solution into a Teflon mold, followed by drying continuously at room temperature for 24 hours to obtain the polysiloxane film;
wherein a stoichiometric ratio of the APDMS, the MDI, the IPDI, and the 2-aminophenylboronic acid is 1:0.6:1.4:2.

2. The application of the siloxane nanocomposite material as claimed in claim 1, wherein a particle size of the nano-silver is 100 nanometers.

3. The application of the siloxane nanocomposite material as claimed in claim 1, wherein the stirring for reaction comprises:
stirring in dark at normal temperature and pressure for 12-24 hours with a stirring speed of 400-500 revolutions per minute.

4. The application of the siloxane nanocomposite material as claimed in claim 1, wherein the adding a polysiloxane film to the suspension and stirring for reaction to obtain the siloxane nanocomposite material comprises:
adding the polysiloxane film to the suspension and stirring for reaction to obtain a reaction product, and air-drying the reaction product in dark at room temperature for 0.5-1 hour to obtain the siloxane nanocomposite material.
